(19) United States
(12) Patent Application Publication
(10) Pub. No.: US 2006/0194967 A1
(43) Pub. Date: Aug. 31, 2006

(54) Process for the preparation of Fenoldopam Mesylate

(76) Inventors: Ettore Bigatti, Balerna (CH); Pierluigi Rossetto, Balerna (CH); Peter Lindsay MacDonald, Gentilino (CH)

Correspondence Address: KENYON & KENYON LLP, ONE BROADWAY, NEW YORK, NY, 10004, US

(21) Appl. No.: 11/339,091

(22) Filed: Jan. 24, 2006

Related U.S. Application Data
(60) Provisional application No. 60/646,942, filed on Jan. 24, 2005.
(60) Provisional application No. 60/649,801, filed on Feb. 3, 2005.
(60) Provisional application No. 60/670,419, filed on Apr. 11, 2005.

(51) Int. Cl. C07D 223/16

(52) U.S. Cl. 540/595; 564/388

(57) ABSTRACT

Methods and intermediates for the preparation of Fenoldopam mesylate and intermediates thereof are provided.

### RELATED APPLICATIONS

This application claims the benefit of U.S. provisional application No. 60/646,942, filed Jan. 24, 2005; Ser. No. 60/649,801, filed Feb. 3, 2005 and Ser. No. 60/670,419, filed Apr. 11, 2005, hereby incorporated by reference.

### FIELD OF THE INVENTION

The present invention is directed to methods for the production of Fenoldopam mesylate and intermediates thereof.

### BACKGROUND OF THE INVENTION

Fenoldopam Mesylate, having the chemical name 6-Chloro-2,3,4,5-tetrahydro-1-(p-hydroxyphenyl)-1H-3-benzazepine-7,8-diol-methanesulfonic acid, and the following structure 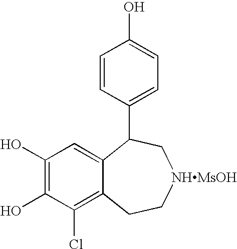
is a rapid-acting vasodilator with agonist effects on dopamine D₁-like receptors, and only moderate affinity for the alpha₂-adrenergic receptors. It is a racemic mixture, with the R-isomer having an approximate 250-fold higher affinity for D₁-like receptors than the S-isomer. Fenoldopam has no agonist effect on presynaptic D₂-like dopamine receptors or on alpha- or beta-adrenergic receptors, and does not appear to affect angiotensin-converting enzyme activity. In animals, Fenoldopam dilates coronary, renal, mesenteric, and peripheral arteries, although vasodilation is not equal in all vascular beds. In normal and hypertensive patients, Fenoldopam appears to dilate renal efferent and afferent arterioles, thereby increasing renal blood flow. However, a beneficial clinical effect on renal function in patients with heart failure or hepatic or severe renal disease has not been demonstrated.

The synthesis for the preparation of Fenoldopam Mesylate (referred to as FM) involves a key step of alkylation of a primary amine. The synthesis disclosed in U.S. Pat. No. 4,197,297 and in U.S. Pat. No. 4,171,359 is conducted by heating equimolar amounts of a styrene oxide with a 3,4-dialkoxy-2-halophenethylamine, to obtain the key intermediate, intermediate III.

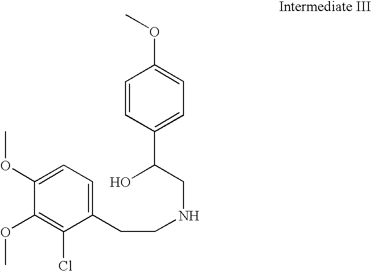

These patents disclose the direct conversion of 2 chloro 3,4 dimethoxyphenethylamine, i.e., 2 chlorohomoveratrylamine, into Intermediate III by condensation with p-methoxystyrene oxide. However, Intermediate III is obtained in only 19% yield.

The synthesis disclosed in EP 0125053 comprises the condensation of p-methoxy-phenylglyoxal methyl hemimercaptal with 2-chlorohomoveratrylamine, followed by the reduction of the carbonyl function and the thioether of resulting compound to obtain the desired intermediate III. However, the preparation of the starting p-methoxy-phenylglyoxal methyl hemimercaptal is complicated and time consuming.

U.S. Pat. No. 5,292,521 discloses a synthetic method comprising the use of methyl mandelate for condensation with 2-chlorohomoveratrylaamine. However, the preparation of Methyl mandelamide, an intermediate in the process requires a diborane reduction step, which is non-selective, i.e. leading to at least one side product, after the condensation. After condensation with 2-chlorohomoveratrylamine, an additional reduction step is necessary in order to obtain Intermediate III, which is obtained in 40% yield.

Weinstock et al., J. MED. CHEM., 1986, 29, 2315, discloses a synthetic method comprising the use of 2-tert-butoxy-1-bromo-p-methoxystyrene in place of p-methoxystyrene oxide. However, the preparation of 2-tert-butoxy-1-bromo-p-methoxystyrene requires four steps from commercially available starting materials. After condensation with 2-chlorohomoveratrylamine, an additional reduction step is necessary in order to obtain Intermediate III.

Therefore, an alternative synthesis from readily available starting materials that over comeovercome the limitations of the known processes would be desirable.

### SUMMARY OF THE INVENTION

In one aspect, the present invention provides a process for the preparation of intermediate II of Fenoldopam of the formula 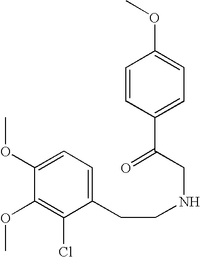
by an alkylation reaction of 2-chlorohomoveratrylamine of formula I,

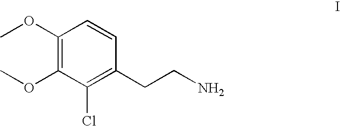

with no more than ⅓ mole equivalents of 2-halo-4′-methoxyacetophenone of formula II 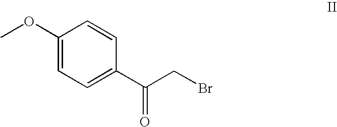 per mole equivalent of the free base of formula I, 2-chlorohomoveratrylamine of formula I.

In another aspect, the present invention provides a process for the preparation of Fenoldopam Mesylate by preparing the intermediate II of Fenoldopam, as described by the process of the present invention, and flurther converting it to Fenoldopam Mesylate.

In yet another aspect, the present invention provides a process for the preparation of the salt of intermediate H of formula II-s of the structure, 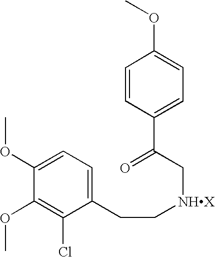
comprising combining intermediate II of Fenoldopam dissolved in a water immiscible organic solvent with a strong acid, in the presence of water; wherein, X is a strong acid, preferably, X is HBr.

In one aspect, the present invention provides a process for purifying the intermediate II of Fenoldopam by combining intermediate II of Fenoldopam dissolved in a water immiscible organic solvent with a strong acid, in the presence of water, to obtain a precipitate of the salt, and adding a base to obtain back the intermediate of formula II.

In another aspect, the present invention provides the novel intermediate II and salts thereof.

In yet another aspect, the present invention provides crystalline salt of intermediate II.

When X is HBr, said compound of formula II-s corresponds to hydrobromide salt of intermediate II of the structure.

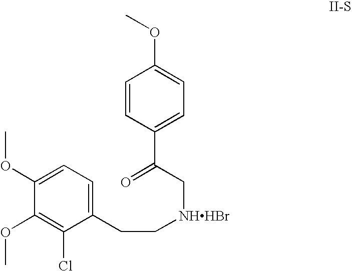

In one aspect, the present invention provides a process for the preparation of Fenoldopam Mesylate by preparing the salt of intermediate II of Fenoldopam, as described by the process of the present invention, and further converting it to Fenoldopam Mesylate.

In another aspect, the present invention provides a process for preparing the salt of intermediate II of formula II-s, comprising alkylating 2-chlorohomoveratrylamine of formula I with no more than ⅓ equivalents of 2-halo-4′-methoxyacetophenone of formula II per mole equivalent of 2-chlorohomoveratrylamine of formula I, and adding a strong acid in the presence of water and water immiscible organic solvent.

In yet another aspect, the present invention provides a process for the preparation of intermediate III of Fenoldopam of the structure In yet another aspect, the present invention provides a process for the preparation of intermediate III of Fenoldopam of the structure 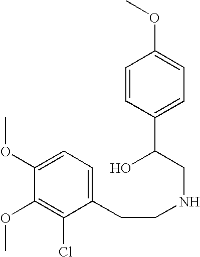
by reducing the intermediate II of Fenoldopam or salts thereof with a reducing agent.

In one aspect, the present invention provides a process for the preparation of Fenoldopam Mesylate by preparing the intermediate III of Fenoldopam, as described by the process of the present invention, and further converting it to Fenoldopam Mesylate.

In another aspect, the present invention provides a process for the preparation of intermediate of formula III of Fenoldopam, comprising alkylating 2-chlorohomoveratrylamine of formula I with no more than ⅓ mole equivalents of 2-halo-4′-methoxyacetophenone of formula II per mole equivalent of 2-chlorohomoveratrylamine of formula I, and adding a strong acid in the presence of water and water immiscible organic solvent; reducing with a reducing agent, and recovering the intermediate of formula III of Fenoldopam.

### BRIEF DESCRIPTION OF THE DRAWINGS

FIG. 1 illustrates a 2-dimensional ¹H-NMR long range C—H coupling spectrum of HBr salt of Fenoldopam Intermediate II.

FIG. 2 illustrates a detailed 2-dimensional ¹H-NMR C—H coupling spectrum of Fenoldopam Intermediate II HBr;

FIG. 3 illustrates a detailed 2-dimensional ¹H-NMR C—H coupling spectrum of Fenoldopam Intermediate II HBr;

FIG. 4 illustrates a mass spectrum of Fenoldopam Intermediate II HBr;

FIG. 5 illustrates an ¹H-NMR spectrum of Fenoldopam Intermediate III HBr;

FIG. 6 illustrates an ¹³C-NMR spectrum of Fenoldopam Intermediate III HBr; and

FIG. 7 illustrates a mass spectrum of Fenoldopam Intermediate III HBr

### DETAILED DESCRIPTION OF THE INVENTION

Alkylation reactions of primary amines are usually accompanied by extensive dialkylated and trialkylated by-products. However, the process of the present invention affords the intermediate II of Fenoldopam

Alkylation reactions of primary amines are usually accompanied by extensive dialkylated and trialkylated by-products. However, the process of the present invention affords the intermediate II of Fenoldopam 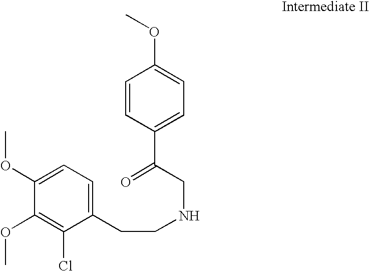
via a successful monoalkylation reaction by using three mole equivalents of the 2-chlorohomoveratrylamine of formula I per mole equivalent of the alkylhalide, leading to a ratio of about 9:1 in favor of the monoalkylated product. After completion of the reaction, the product is recovered in a pure state, as a salt of the monoalkylated product, by an easy separation from the excess of the starting amine, while recycling the expensive starting amine of formula I.

The present invention provides a process for the preparation of intermediate II of Fenoldopam of the formula by an alkylation reaction of 2-chlorohomoveratrylamine of formula I. 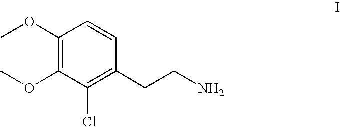
with no more than ⅓ mole equivalents of 2-halo-4′-methoxyacetophenone of formula II 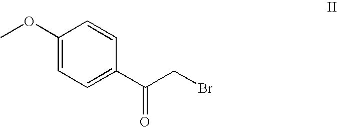
per mole equivalent of 2-chlorohomoveratrylamine of formula I.

The alkylation reaction is performed on the free base, 2-chlorohomoveratrylamine of formula I, which is available as a salt, for example, according to the process disclosed in J. Med.Chem. 1986, 29, 1586. Prior to performing the alkylation reaction, the base is liberated, for example, by combining the salt of 2-chlorohomoveratrylamine of formula I, preferably, the hydrobromide salt, with a mixture of C₁₋₂ halogenated hydrocarbon and water, and with a base, preferably, sodium hydroxide, or by any method known in the art.

The free base may be used in the alkylation step, without further purification.

Preferably, the alkylation is done by combining, at a temperature of about 10° C. to about 0° C., the free base of formula I with water immiscible organic solvent and with no more than ⅓ mole equivalents of 2-halo-4′-methoxyacetophenone of formula II per mole equivalent of the free base of formula I, to obtain a mixture. The mixture is maintained for about 5 to about 200 minutes, to obtain a precipitate.

Preferably, 2-halo-4′-methoxyacetophenone of formula II is selected from 2-chloro-4′-methoxyacetophenone, 2-bromo-4′-methoxyacetophenone and 2-iodo-4′-methoxyacetophenone. More preferably, 2-halo-4′-methoxyacetophenone of formula II is 2-bromo-4′-methoxyacetophenone.

Preferably, the free base of formula I is used in an amount of about 3 to about 5 mole equivalents per mole equivalent of the 2-halo-4′-methoxyacetophenone of formula II, more preferably, of about 3 to about 3.3 mole equivalents, and even more preferably, of about 3 mole equivalents.

Preferably, the water immiscible organic solvent is selected from a group consisting of C₁₋₂ halogenated hydrocarbon, C₁₋₁₂ aliphatic hydrocarbon, ether and C₆₋₈ aromatic hydrocarbon. A preferred C₁₋₂ halogenated hydrocarbon is C₁₋₂ chlorinated hydrocarbon, more preferably, dichloromethane (referred to as DCM), dichloroethane or chloroform. Preferably, the C₁₋₁₂ aliphatic hydrocarbon is either hexane or heptane. A preferred ether is diethylether. Preferably, the C₆₋₈ aromatic hydrocarbon is toluene. Most preferably, the water immiscible organic solvent is DCM.

Preferably, the free base of formula I is combined, first, with the solvent and only then, the 2-halo-4′-methoxyacetophenone of formula II is added, thus maintaining a large excess of the free base of formula I in the mixture.

Preferably, the reacting substances are combined at a temperature of about 5° C. to about 0° C., more preferably, at a temperature of about 2° C. to about 0° C.

Preferably, the mixture is agitated during the reaction.

Preferably, the mixture is maintained for about 10 to about 100 minutes, more preferably, for about 15 minutes.

The present invention further provides a process for the preparation of Fenoldopam Mesylate by preparing the intermediate II of Fenoldopaam, as described by the process of the present invention, and further converting it to Fenoldopam Mesylate.

The present invention also provides a process for the preparation of the salt of intermediate II of formula II-s of the structure, 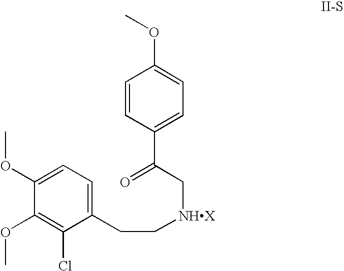
comprising combining intermediate II of Fenoldopam dissolved in water immiscible organic solvent, with a strong acid, in the presence of water; wherein, X is a strong acid preferably, X is HBr.

Preferably, the water immiscible organic solvent is the same as those that are used in the alkylation reaction.

Preferably, the strong acid is selected from a group consisting of methanesulfonic acid, hydrochloric acid, perchloric acid, sulfuric acid, hydrobromic acid and phosphoric acid. More preferably, the strong acid is HBr.

Preferably, the concentration of HBr is of about 48% or less, more preferably, of about 3.65% to about 48%, whereby, dilution can be obtained by the addition of water.

The salt may be obtained, preferably, by cooling the mixture obtained by combining intermediate II of Fenoldopam dissolved in a water immiscible organic solvent with a strong acid, in the presence of water, to a temperature of about 20° C. to about 0° C., more preferably, to about 10° C. to about 0° C., and even more preferably, to about 4° C. to about 2° C., forming a precipitate.

The salt may be recovered by collecting the precipitate, preferably, by using a filter and a vacuum pump, followed by washing the precipitate with water and with water immiscible organic solvent, and drying in a vacuum oven. Preferably, the salt is recovered in purity of about 92% to about 100%, more preferably, of about 95% to about 100%, and even more preferably, of about 96%.

The expensive unreacted 2-chlorohomoveratrylamine of formula I may be recycled from the filtrate obtained from filtering the precipitate of the salt. Accordingly, the two phases comprising the filtrate are separated, and the aqueous phase is combined with a new portion of water immiscible organic solvent and with a base, preferably, sodium hydroxide. After the pH is adjusted to about 10 to about 11, preferably, to about 10 to about 10.5, the phases are separated again, and the organic phase is concentrated providing 2-chlorohomoveratrylamine of formula I.

The present invention further provides a process for purifying the intermediate II of Fenoldopam by combining intermediate II of Fenoldopam dissolved in water immiscible organic solvent with a strong acid, in the presence of water, to obtain a precipitate of the salt, and adding a base to obtain back the intermediate of formula II.

Preferably, the precipitate of the salt is recovered, and then it is reacted with the base.

The present invention also provides the novel intermediate II and salts thereof.

The present invention provides crystalline salt of intermediate II of formula II-s.

When X is HBr, said compound of formula I-s corresponds to hydrobromide salt of intermediate II of the structure. 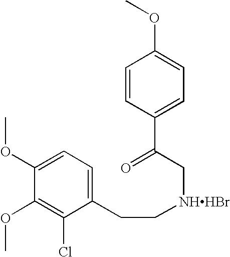
The hydrobromide salt of intermediate II of Fenoldopam may be characterized by data selected from: a melting temperature of about 205.2° C.; by an ₁H-NMR (DMSOd₆, 75 MHz) spectrum having peaks at about 3.15, 3.75, 3.83, 3.89, 4.82, 7.06, 7.13, 7.14, 8.00 and 9.11 ppm, by a ¹³C-NMR (DMSOd₆, 300 MHz) spectrum having peaks at about 29, 46.3, 51.9, 55.7, 57, 60.0, 111.6, 114.3, 125.4, 126.4, 127.2, 127.3, 130.6, 144.9, 154.2, 164.2 and 190.5 ppm, and by Mass spectrum having peaks at about (ESI⁺) MH⁺364.

The present invention further provides a process for the preparation of Fenoldopam Mesylate by preparing the salt intermediate II of Fenoldopam, as described by the process of the present invention, and further converting it to Fenoldopam Mesylate.

The present invention also provides a process for preparing the salt of intermediate II of formula I-s, comprising alkylating 2-chlorohomoveratrylamine of formula I with no more than ⅓ mole equivalents of 2-halo-4′-methoxyacetophenone of formula II per mole equivalent of the free base of formula I, and adding a strong acid in the presence of water and water immiscible organic solvent.

The process to obtain the salt of the intermediate II of Fenoldopam may be run stepwise or concurrently, i.e., without isolation of the intermediate II of Fenoldopam prior to the preparation of the salt. Preferably, the process is run concurrently.

The salt of the intermediate II of Fenoldopam may be used in the reduction step, without further purification.

The present invention provides a process for the preparation of intermediate III of Fenoldopam of the structure 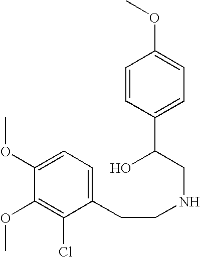
by reducing the intermediate II of Fenoldopam or salts thereof with a reducing agent.

Optionally, the salt of the intermediate II of Fenoldopam may be converted to the free base, the intermediate II of Fenoldopam, prior to performing the reduction step. The conversion may be done by reacting a mixture of the salt of intermediate II of Fenoldopam in a mixture of water immiscible organic solvent and water with a base, to obtain complete dissolution of the salt. After complete dissolution has occurred, two phases are obtained and separated.

Preferably, the conversion is done under stirring.

Preferably, the base can be either organic or inorganic. Preferred inorganic bases include potassium hydroxide, lithium hydroxide, sodium hydroxide, and ammonium hydroxide. A preferred organic base is either triethylamine or tributylamine. The more preferred base is ammonium hydroxide.

The free base, the intermediate II of Fenoldopam, may be used for the reduction step without isolation, i.e. dissolved in the organic phase, obtained after the separation of the phases.

The reduction may be done, preferably, by combining the intermediate II of Fenoldopam or salts thereof with a mixture of water immiscible organic solvent and C₁₋₄ alchohol, and with a reducing agent, to obtain a mixture. The mixture is then maintained for about 90 to about 120 minutes, followed by recovering the intermediate III of Fenoldopam.

Preferably, the water immiscible organic solvent is the same as those that are used in the alkylation reaction. Preferably, the C₁₋₄ alchohol is methanol, ethanol, propanol, butanol or isopropanol, more preferably, methanol.

Preferably, the mixture is maintained under stirring.

Preferably, the reducing agent is selected from a group consisting of metal hydride, more preferably, LiAlH₄, sodium cyanoborohydride or NaBH₄, and even more preferably, NaBH₄.

The progress of the reaction may be monitored by HPLC.

The intermediate III of Fenoldopam may be recovered by combining the mixture with water, followed by agitating for about 5 to about 20 minutes, preferably, for about 15 minutes, and separating the organic phase, which is concentrated, to give a residue. The residue is then combined with a solvent selected from a group consisting of C₁₋₆ ester, C₁₋₆ ketone and mixtures of C₁₋₆ ketone and C₁₋₆ aliphatic hydrocarbon, followed by evaporating the solvent, and adding a second portion of a C₁₋₆ ester forming a suspension. The suspension is agitated, preferably, at atmospheric pressure and under heating, for about 20 minutes to about an hour, and then, cooled to a temperature of about 0° C. to about 5° C. for about 20 minutes to about an hour to precipitate intermediate III of Fenoldopam. The precipitate is then collected by filtration and washed by a C₁₋₆ ester, preferably, maintained at a temperature of about 0° C. to about 5° C., followed by drying in a vacuum oven.

Preferably, the C₁₋₆ ester is ethylacetate, methylacetate, isopropylacetate or ethylpropionate. A preferred C₁₋₆ Ketone is either acetone or methyl ethyl ketone. Preferably, the C₁₋₆ aliphatic hydrocarbon is hexane. More preferably, the solvent is C₁₋₆ ester, most preferably, ethylacetate.

The suspension is agitated, preferably, for about 30 minutes.

Preferably, the cooling is done for over 30 minutes.

The process of the present invention for the preparation the intermediate III of Fenoldopam is found to be cost effective and efficient and can also be adapted to industrial scale, as compared to the processes of the prior art, that are not suitable for production purposes, and are not as efficient and cost effective as the process of the present invention

The present invention further provides a process for the preparation of intermediate of formula III of Fenoldopam, comprising alkylating 2-chlorohomoveratrylamine of formula I with no more than ⅓ mole equivalents of 2-halo-4′-methoxyacetophenone of formula II per mole equivalent of 2-chlorohomoveratrylamine of formula I, and adding a strong acid in the presence of water and a water immiscible organic solvent; reducing with a reducing agent, and recovering the intermediate of formula III of Fenoldopam.

The present invention also provides a process for the preparation of Fenoldopam Mesylate by preparing the intermediate III of Fenoldopam, as described by the process of the present invention, and further converting it to Fenoldopam Mesylate, for example, by the process disclosed in U.S. Pat. No. 4,171,359. Accordingly, the intermediate III of Fenoldopam may be converted to Fenoldopam Mesylate, by cyclizing it in the presence of a mixture of trifluoroacetic acid, concentrated sulfuric acid and water, to obtain Fenoldopam trimethyl ether, which is then reacted with hydrobromic acid in the presence of phenol, forming the hydrobromide salt of Fenoldopam, and transforming Fenoldopam hydrobromide to Fenoldopam Mesylate by liberating the free base, followed by salt formation with methane sulfonic acid.

### EXAMPLES

The process for the manufacture of 2-chloroisovanillin used in the invention is based upon that disclosed by Faulkner and Woodcock, JOURNAL OF THE CHEMICAL SOCIETY, 1962, 4737, the teachings of which are incorporated herein in their entirety. However, rather than chloroform, the chlorination step is performed in a dioxane solvent. In addition, in the invention, the process for the manufacture of 2-chloro-3,4-dimethoxybenzaldehyde is based upon that disclosed in U.S. Pat. No. 4,160,765, the teachings of which are incorporated herein in their entirety.

The process of the invention for the manufacture of 2-chloro-3,4-dimethoxy-p-nitrostyrene is based upon that disclosed in U.S. Pat. No. 4,160,765.

Reduction of 2-chloro-3,4-dimethoxy-B-nitrostyrene using lithium aluminum hydride is disclosed U.S. Pat. No. 4,108,989, the teachings of which are incorporated herein in their entirety. In an improved procedure disclosed in J. MED. CHEM., 1986, Vol 29, 1586, the teachings of which are incorporated herein in their entirety, the hydride reduction is conducted in the presence of dry aluminum chloride. The modified procedure leads to a purer reaction product in higher yield, and forms the basis for the method of the present invention.

HPLC

The analysis is using a standard RP-18 250*4.6 mm column, using an eluent flow of 2 ml/m of a mixture of 25% 0.013% H₃PO₄ aqueous solution and 75% acetonitrile, 225 mn UV revelator, the product is eluted in about 10 minutes with 10% less retention time of intermediate III.

### Example 1

### Production of 2-Chlorohomoveratrylamine Freebase

A glass reactor was charged with 500 grams of 2-chlorohomoveratrylamine hydrobromide, 1820 grams of distilled apyrogenic water 1820 grams of dichloromethane, and agitated. The pH of the aqueous phase was adjusted to at least about 10 with 221 grams of a 32 percent by weight sodium hydroxide solution. The two phases were allowed to separate, and the organic phase was drawn off into a tared Büchi flask. The solvent was evaporated at atmospheric pressure, using a bath temperature of about 60° C., to provide an oily residue (100% yield). The 2-chlorohomoveratrylamine freebase was dissolved the in 3160 grams of dichloromethane.

### Example 2

### Preparation of Fenoldopam Intermediate II by Condensation

A glass reactor was charged with the 2-chlorohomoveratrylamine freebase obtained in Example 1. The freebase was agitated and cooled to between about 0° C. and about 2° C. The glass reactor was then charged additionally with 129 grams of 2-bromo-4′-methoxyacetophenone. After about 15 minutes, a precipitate formed, and the contents of the reactor were agitated for an additional 40 to 50 minutes at about 0° C. to about 5° C.

### Example 3

### Isolation of Fenoldopam Intermediate II Hydrobromide

The glass reactor of Example 2 was charged additionally with a diluted solution of 237 grams of 48 percent hydrobromic acid in 3000 grams of distilled apyrogenic water. The resulting mixture was agitated, and cooled to between about 2° C. and about 4° C. The resulting precipitate was collected using a Büchner filter and a vacuum pump compatible with acid vapors, and rinsed, first with 1290 grams of distilled apyrogenic water, and then with 322 grams of dichloromethane. The vacuum was applied until the mother liquors were no longer being removed. A small sample was dried in oven at 60° C. under vacuum, and a melting point of 205.2° C. was determined. Purity as determined by HPLC: 96%. When the entire product is dried about 182 grams of intermediate II was obtained (73% yield).

### Example 4

### Recovery of 2-Chlorohomoveratrylamine

The precipitate of Intermediate II produced in Example 3 was used without drying. The filtrates from Example 3 were charged into a glass reactor, and the two phases were allowed to separate. The lower organic phase was drawn off, and discarded. The reactor was then charged with 1390 grams of dichloromethane, agitated, and the pH was adjusted to at least about 10 with 199 grams of a 32 percent sodium hydroxide solution. The two phases were allowed to separate, and the lower organic phase into a tared Büchi flask. Most of the solvent was evaporated at atmospheric pressure with a bath temperature of about 60° C., and a vacuum was then applied at a pressure of no more than about 50 mbar for about 30 minutes, recovering 236 grams of the excess 2-chlorohomoveratrylamine as an oily residue.

### Example 5

### Reduction of Intermediate II

A glass reactor was charged with the fenoldopam Intermediate II hydrobromide, isolated in Example 3, 2728 grams of dichloromethane, and 1820 grams of distilled apyrogenic water, and agitated. The reactor was then charged with 72.7 grams of a 25 percent solution of ammonium hydroxide solution, and the agitation was continued until the dissolution of the hydrobromide was complete. The two phases were allowed to separate. The lower organic phase was drawn off into a flask, and the upper aqueous phase was discarded.

The organic phase was returned into the reactor, and 15.2 grams of sodium borohydride and 910 grams of methanol were added. The mixture was stirred for about 90 to about 120 minutes until reduction was complete (HPLC control, method 9062M.M, limit 0.5% remaining Intermediate II).

Isolation of Fenoldopam Intermediate III

The glass reactor from Example 5 was charged with 2270 grams of distilled apyrogenic water, and the contents were agitated for about 15 minutes. The phases were allowed to separate, and the lower organic phase was drawn off. The solution was evaporated in a Büchi flask in a Rotovapor (atmospheric pressure/bath temperature 60° C.) to a semicrystalline residue.

The Büchi flask was then charged with 545 grams of ethyl acetate, and the solution was again evaporated in the Rotovapor at a pressure of no more than about 50 mbar and a bath temperature of 60° C. to a semicrystalline residue. The Büchi flask was then charged with 364 grams of ethyl acetate, and the resulting suspension was agitated on the Rotovapor (atmospheric pressure/bath temperature 60° C.) for about 30 minutes until a filterable suspension was obtained.

The contents of the Büchi flask were cooled to about 0° C. in an ice bath for about 30 minutes, and the precipitate of Intermediate III was collected on Büchner filter, rinsing with 127 grams of ethyl acetate, previously cooled to about 0° C. in an ice bath. The collected crystals of Intermediate III were dried in a vacuum oven at about 55° C. to about 60° C. for about 12 to about 16 hours obtaining 133 grams with a HPLC purity of 99.5% (89% yield of reduction) (overall yield 65% from 2-bromo-4′-methoxyacetophenone).

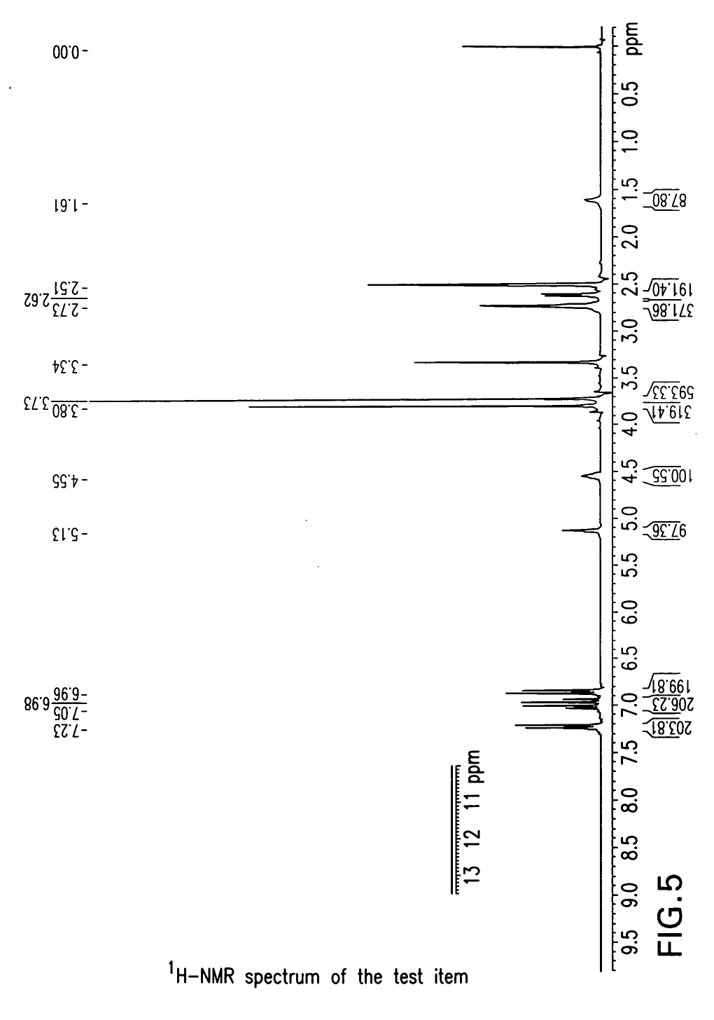

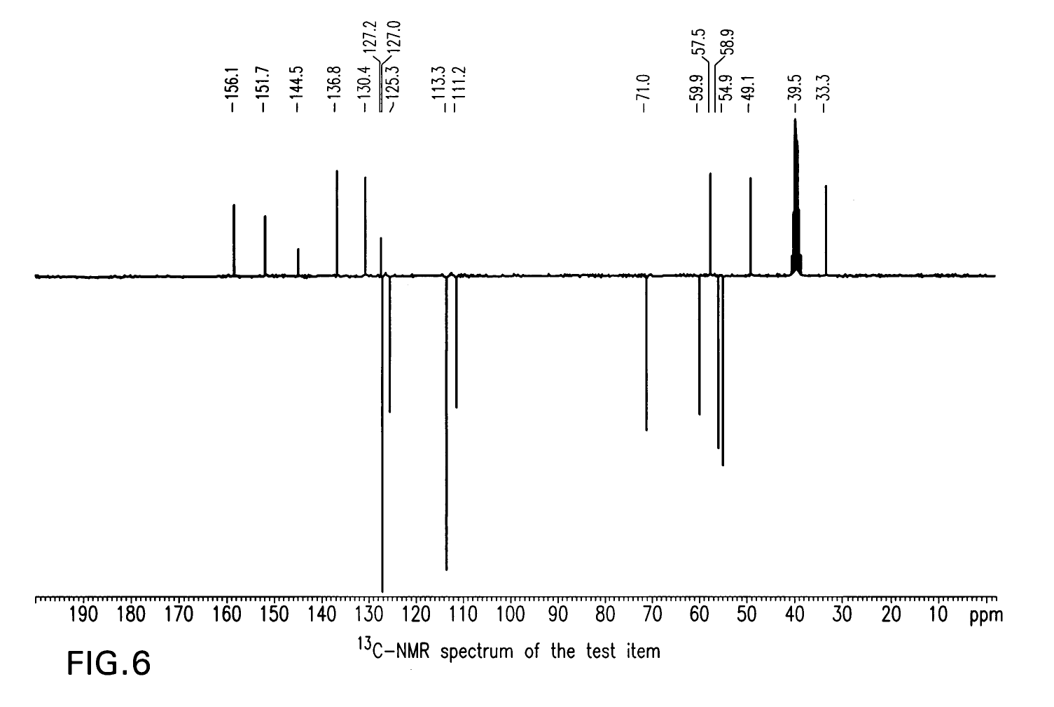

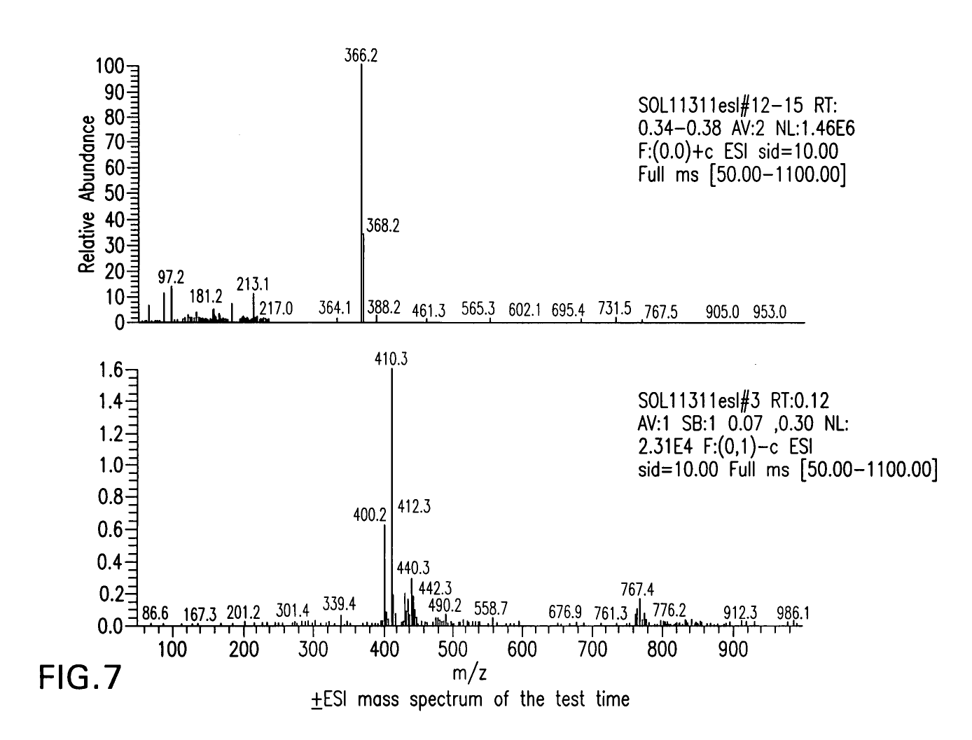

The Intermediate II and III products were characterized using ¹H-NMR and ¹³C-NMR and mass spectrometry. The results are provided in FIGS. 3 to 11.

### Example 6

### Preparation of Fenoldopam Hydrobromide

A 3 liter glass reactor was charged with 106 grams of Fenoldopam Intermediate III, as isolated in Example 6, 530 grams of dichloromethane, and agitated at about 0° C. To the agitated mixture, 265 grams of trifluoroacetic acid were added, followed by 42 grams of concentrated sulfuric acid. After 2 hours, when the reaction was complete, 1500 grams of distilled apyrogenic water and 500 grams of dichloromethane were added, followed by 285 grams of 25 percent by weight ammonium hydroxide solution, while the mixture was maintained at room temperature with cooling.

After complete extraction, the separated organic phase was evaporated in a 2 liter flask to an oily residue. Then, 106 grams of phenol were added, and the solvent was again evaporated to eliminate traces of dichloromethane. Following the evaporation, 1166 grams of 48 percent hydrobromic acid were added. The mixture was then heated to about 107° C. with low sparging of nitrogen, and stirred continuously at that temperature for 16 hours.

The mixture was then cooled, and 477 grams of tetrahydrofuran were added. After 30 minutes at room temperature, the mixture was filtered on a Büchner funnel, the precipitate was rinsed with 477 grams of ethyl acetate, and dried at 60° C. under vacuum for 3 hours, providing 82.4 grams of the product. (73.5% yield).

### Example 7

### Preparation of Fenoldopam Mesylate

A 2 liter glass reactor was charged with 79 grams of Fenoldopam hydrobromide, 790 grams of methanol, and stirred and under nitrogen for 30 minutes. A solution of 18 grams of sodium bicarbonate in 345 grams of distilled apyrogenic water was added. The mixture was stirred for 15 minutes at room temperature, cooled to 5° C., and filtered on a Büchner funnel. The precipitate was rinsed with 400 grams of distilled apyrogenic water, and transferred to another flask together with 790 grams of methanol. The resulting mixture was acidified with 19 grams of methanesulfonic acid to a pH of 2.5. After clarification, the solution was evaporated to about 400 grams under vacuum, 400 grams of water were added, and the mixture was again evaporated to 333 grams.

After 16 hours of stirring at room temperature, the precipitate was filtered on a Büchner funnel, and washed, first with 40 grams of distilled apyrogenic water, and then with 160 grams of isopropanol. After 16 hours of drying at 60° C. under vacuum, 70.3 grams of fenoldopam mesylate (85.9% yield), having an HPLC purity of 99.4 percent, was obtained.

### Example 8

### Purification of Fenoldopam Mesylate

A solution of 100 grams of Fenoldopam mesylate, of the type prepared in example 8, was dissolved in a mixture of 6300 grams of distilled apyrogenic water, 700 grams of methanol, and an amount of methanesulfonic acid q.b., sufficient to provide a pH of 2.2, was charged onto a 40×300 mm C-18 CARTRIDGE, and eluted with about 7 liters of 12 percent by weight methanolic acid solution.

The fractions having a purity greater than 99 percent were pooled, concentrated, and crystallized as described in example 8. The product was triturated with 1600 grams of hot isopropanol, cooled, filtered again, and finally dried at 80° under vacuum for 16 hours. As a result, 87 grams of a non-hygroscopic crystalline, first crop of product, having an HPLC purity of 100 percent was obtained.

While it is apparent that the invention disclosed herein is well calculated to fulfill the objects stated above, it will be appreciated that numerous modifications and embodiments may be devised by those skilled in the art. Therefore, it is intended that the appended claims cover all such modifications and embodiments as falling within the true spirit and scope of the present invention.

What is claimed:

1. Intermediate II of Fenoldopam of the structure 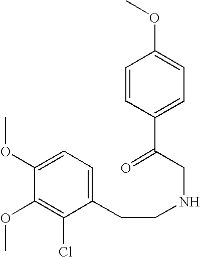
and salts thereof, wherein the salts are of the structure

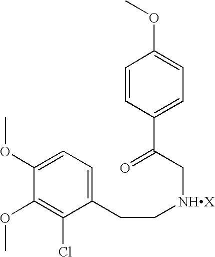

2. Intermediate II of Fenoldopam of claim 1 of the structure

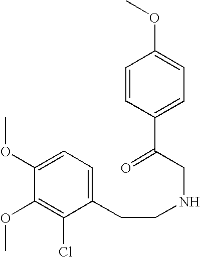

3. Salts of Intermediate II of Fenoldopam of claim 1 of the structure.

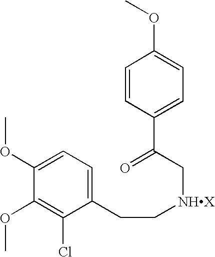

4. The salt of intermediate II of claim 4, wherein X is HBr.

5. The salt of intermediate II of claim 1, characterized by data selected from the group consisting of: a melting temperature of about 205.2° C., an ¹H-NMR spectrum (DMSOd₆, 75 MHz) spectrum having peaks at about 3.15, 3.75, 3.83, 3.89, 4.82, 7.06, 7.13, 7.14, 8.00 and 9.11 ppm, a ¹³C-NMR (DMSOd₆, 300 MHz) spectrum having peaks at about 29, 46.3, 51.9, 55.7, 57, 60.0, 111.6, 114.3, 125.4, 126.4, 127.2, 127.3, 130.6, 144.9, 154.2, 164.2 and 190.5 ppm, and a mass spectrum having peaks at about (ESI⁺) MH⁺364.

6. The salt of intermediate II of claim 5, characterized by a melting temperature of about 205.2° C.

7. The salt of intermediate II of claim 5, characterized by an ¹H-NMR spectrum (DMSOd₆, 75 MHz) spectrum having peaks at about 3.15, 3.75, 3.83, 3.89, 4.82, 7.06, 7.13, 7.14, 8.00 and 9.11 ppm.

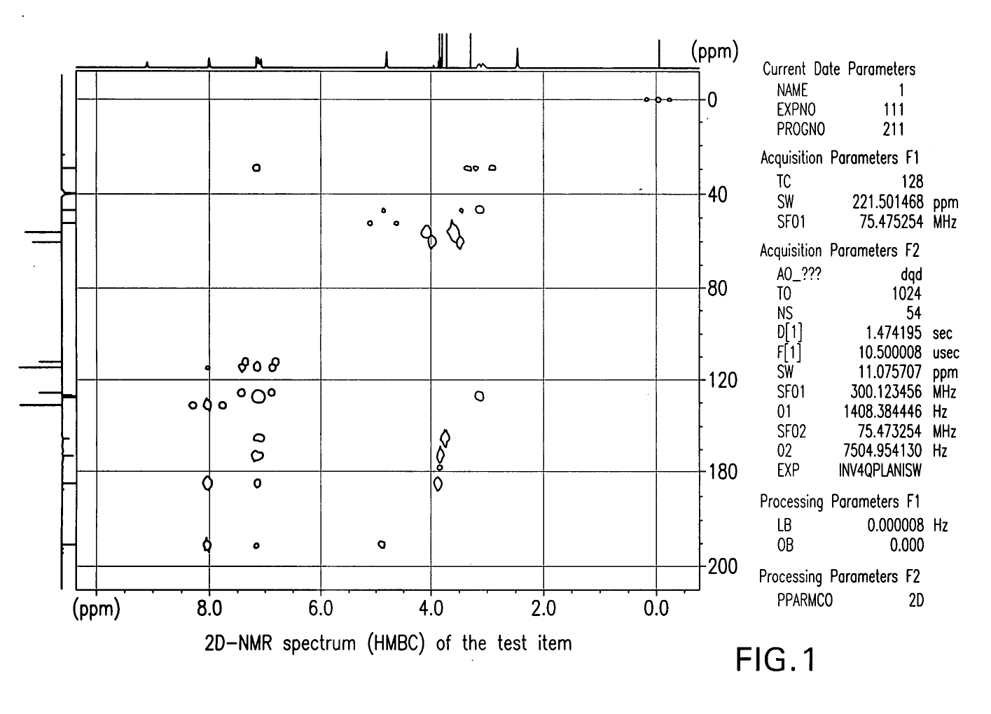

8. The salt of intermediate II of claim 7, having an ¹H-NMR spectrum substantially as depicted in FIG. 1.

9. The salt of intermediate II of claim 5, characterized by a ¹³C-NMR (DMSOd₆, 300 MHz) spectrum having peaks at about 29, 46.3, 51.9, 55.7, 57, 60.0, 111.6, 114.3, 125.4, 126.4, 127.2, 127.3, 130.6, 144.9, 154.2, 164.2 and 190.5 ppm.

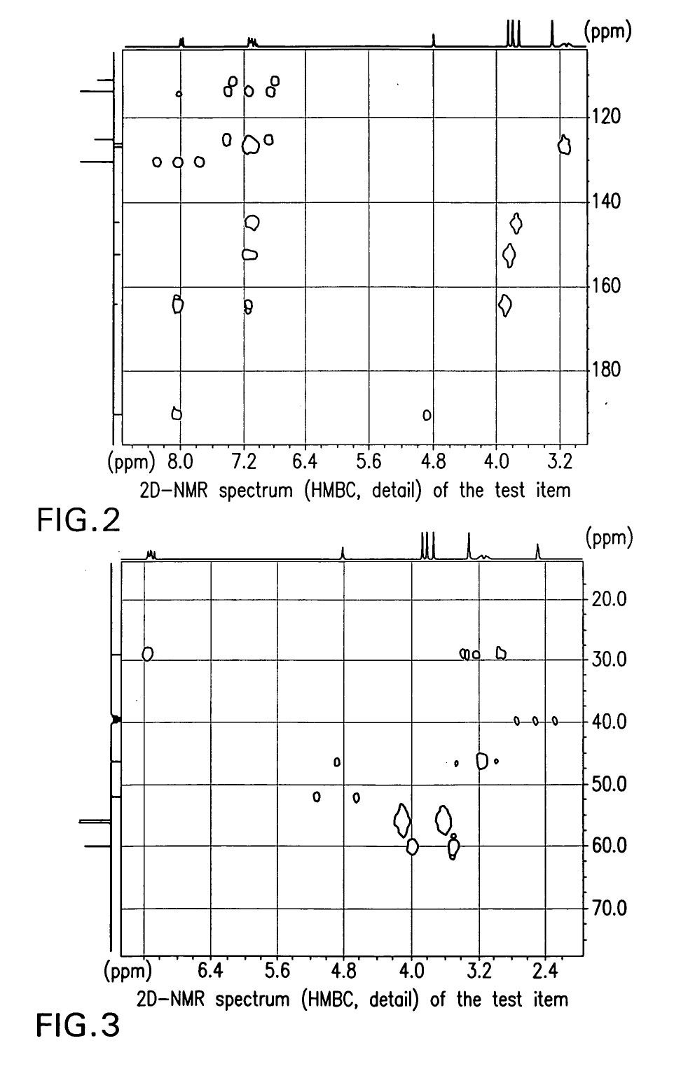

10. The salt of intermediate II of claim 1, having a ¹³C-NMR spectrum substantially as depicted in FIG. 2.

11. The salt of intermediate II of claim 5, characterized by a mass spectrum having peaks at about (ESI⁺) MH⁺364.

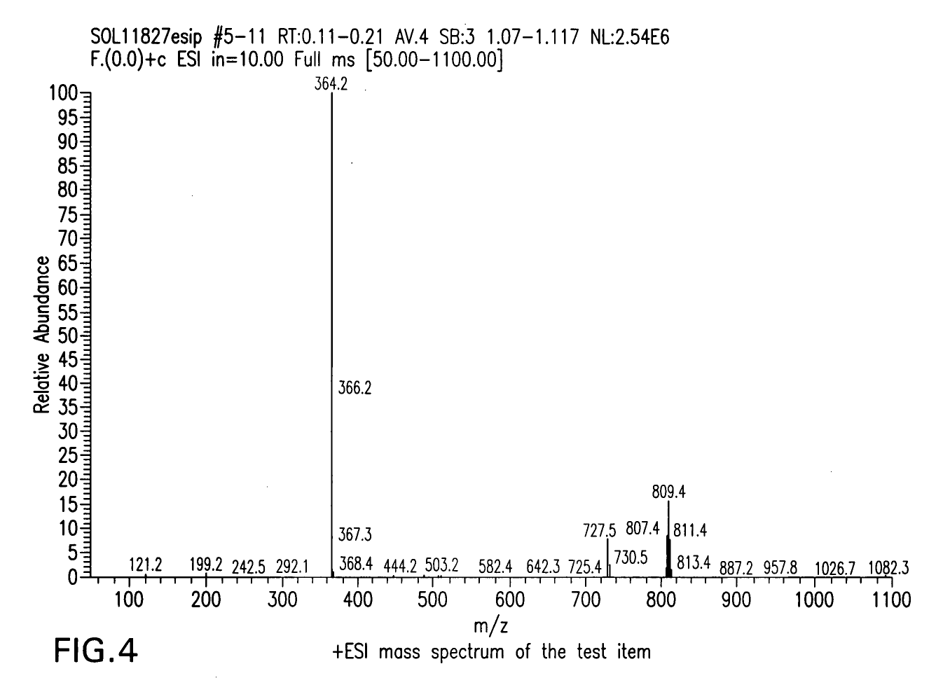

12. The salt of intermediate II of claim 11, having a MS spectrum substantially as depicted in FIG. 4.

13. A crystalline salt of intermediate II of claim 4.

14. A process for preparing the intermediate II of claim 1, comprising an alkylation reaction of 2-chlorohomoveratrylsmine of formula I 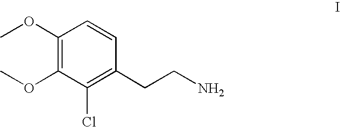
with no more than ⅓ mole equivalents of 2-halo-4′-methoxyacetophenone of formula II 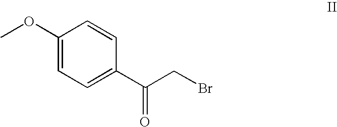
per mole equivalent of 2-chlorohomoveratrylsmine of formula I.

15. The process of claim 14, wherein the alkylation is done by combining, at a temperature of about 10° C. to about 0° C., the free base of formula I with water immiscible organic solvent and with no more than ⅓ mole equivalents of 2-halo-4′-methoxyacetophenone of formula II per mole equivalent of the free base of formula I, to obtain a mixture; and maintaining the mixture for about 5 to about 200 minutes, to obtain a precipitate.

16. The process of claim 15, wherein the free base of formula I is used in an amount of about 3 to about 5 mole equivalents per mole equivalent of the 2-halo-4′-methoxyacetophenone of formula II.

17. The process of claim 16, wherein the free base of formula I is used in an amount of about 3 to about 3.3 mole equivalents per mole equivalent of the 2-halo-4′-methoxyacetophenone of formula II.

18. The process of claim 17, wherein the free base of formula I is used in an amount of about 3 mole equivalents per mole equivalent of the 2-halo-4′-methoxyacetophenone of formula II.

19. The process of claim 15, wherein the water immiscible organic solvent is selected from a group consisting of C₁₋₂ halogenated hydrocarbon, C₁₋₁₂ aliphatic hydrocarbon, ether and C₆₋₈ aromatic hydrocarbon.

20. The process of claim 19, wherein the C₁₋₁₂ aliphatic hydrocarbon is either hexane or heptane.

21. The process of claim 19, wherein the C₁₋₂ halogenated hydrocarbon is C₁₋₂ chlorinated hydrocarbon.

22. The process of claim 21, wherein the C₁₋₂ chlorinated hydrocarbon is dichloromethane, dichloroethane or chloroform.

23. The process of claim 22, wherein the C₁₋₂ chlorinated hydrocarbon is dichloromethane.

24. The process of claim 22, wherein the water immiscible organic solvent is dichloromethane.

25. The process of claim 15, wherein the 2-halo-4′-methoxyacetophenone of formula II is selected from 2-chloro-4′-methoxyacetophenone, 2-bromo-4′-methoxyacetophenone and 2-iodo-4′-methoxyacetophenone.

26. The process of claim 25, wherein the 2-halo-4′-methoxyacetophenone of formula II is 2-bromo-4′-methoxyacetophenone.

27. The process of claim 15, wherein the free base of formula I is combined, first, with the solvent and only then, the 2-halo-4′-methoxyacetophenone of formula II is added.

28. The process of claim 15, wherein the mixture is cooled to a temperature of about 5° C. to about 0° C.

29. The process of claim 28, wherein the mixture is cooled to a temperature of about 2° C. to about 0° C.

30. The process of claim 15, wherein the mixture is agitated during the reaction.

31. The process of claim 15, wherein the mixture is maintained for about 10 to about 100 minutes.

32. A process for preparing fenoldopam mesylate by preparing the compound of claim 2, and converting it to fenoldopam mesylate.

33. A process for the preparation of the salt of Intermediate II, comprising combining the compound of claim 2, dissolved in a water immiscible organic solvent, with an acid, in the presence of water; wherein, X is a strong acid.

34. The process of claim 33, wherein the strong acid is selected from a group consisting of hydrochloric acid, perchloric acid, sulfuric acid, hydrobromic acid and phosphoric acid.

35. The process of claim 34, wherein the strong acid is HBr.

36. The-process of claim 35, wherein the concentration of HBr is of about 48% or less.

37. The process of claim 36, wherein concentration of HBr is of about 3.65% to about 48%.

38. The process of claim 37, whereby dilution can be obtained by the addition of water.

39. The process of claim 33, wherein a mixture is obtained by combining the Intermediate II, dissolved in a water immiscible organic solvent, with an acid in the presence of water.

40. The process of claim 39, wherein the mixture is cooled to a temperature of about 20° C. to about 0° C.

41. The process of claim 40, wherein the mixture is cooled to a temperature of about 10° C. to about 0° C.

42. The process of claim 41, wherein the mixture is cooled to a temperature of about 4° C. to about 2° C.

43. A process for purifying Intermediate II, comprising combining the compound of claim 2, dissolved in a water immiscible organic solvent, with a strong acid in the presence of water to obtain a precipitate of the salt, and adding a base to obtain back the intermediate of formula II.

44. The process of claim 43, wherein the salt is recovered and then is reacted with the base.

45. A process for the preparation of fenoldopam mesylate by preparing the compound of claim 1, and converting it to fenoldopam mesylate.

46. A process for preparing the compound of claim 1, comprising alkylating 2-chlorohomoveratrylsmine of formula I with no more than ⅓ mole equivalents of 2-halo-4′-methoxyacetophenone of formula II per mole equivalent of the free base of formula I, and adding a strong acid in the presence of water and water immiscible organic solvent.

47. The process of claim 46, whereby the process is run concurrently or step-wise.

48. The process of claim 46, whereby the process is run concurrently.

49. The process of claim 46, wherein Intermediate II is used in the reduction step, without further purification.

50. A process for the preparation of intermediate III of Fenoldopam of the structure 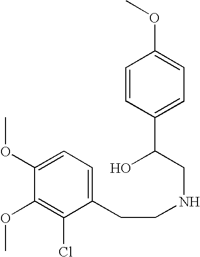
by reducing the intermediate II of Fenoldopam of claim 1 or a salt thereof with a reducing agent.

51. The process of claim 50, wherein the Intermediate II is converted to a salt of Intermediate II prior to performing the reduction step.

52. The process of claim 51, wherein the conversion is done by reacting a mixture of the Intermediate II in a mixture of water immiscible organic solvent and water, with a base, to obtain complete dissolution of the salt, and separating the phases that are obtained.

53. The process of claim 52, wherein the conversion is done under stirring.

54. The process of claim 52, wherein the base is either organic or inorganic.

55. The process of claim 54, wherein the inorganic base is sodium hydroxide.

56. The process of claim 54, wherein the inorganic base is potassium hydroxide, lithium hydroxide, sodium hydroxide or ammonium hydroxide.

57. The process of claim 54, wherein the organic base is either triethylamine or tributylamine.

58. The process of claim 54, wherein the base is ammonium hydroxide.

59. The process of claim 52, wherein Intermediate II is used for the reduction step without isolation.

60. The process of claim 52, wherein the reduction is done by, combining Intermediate II with a mixture of water immiscible organic solvent and C₁₋₄ alchohol, and with a reducing agent, to obtain a mixture; maintaining the mixture for about 90 to about 120 minutes, and recovering the intermediate III of Fenoldopam.

61. The process of claim 60, wherein the mixture is maintained under stirring.

62. The process of claim 60, wherein the reducing agent is selected from a group consisting of a metal hydride complex.

63. The process of claim 62, wherein the reducing agent is a metal hydride.

64. The process of claim 63, wherein the metal hydride is LiAlH₄, sodium cyanoborohydride or NaBH₄.

65. The process of claim 62, wherein the reducing agent is NaBH₄.

66. The process of claim 60, wherein the C₁₋₄ alchohol is methanol, ethanol, propanol, butanol or isopropanol.

67. The process of claim 66, wherein the C₁₋₄ alchohol is methanol.

68. A process for the preparation of intermediate of formula III of Fenoldopam, comprising alkylating 2-chlorohomoveratrylsmine of formula I with no more than ⅓ mole equivalents of 2-halo-4′-methoxyacetophenone of formula II per mole equivalent of 2-chlorohomoveratrylsmine of formula I; adding a strong acid in the presence of water and a water immiscible organic solvent; reducing with a reducing agent, and recovering the intermediate of formula III of Fenoldopam.

69. A process for the preparation of fenoldopam mesylate by preparing intermediate III of Fenoldopam, and converting it to fenoldopam mesylate.
